(19) United States
(12) Patent Application Publication
(10) Pub. No.: US 2014/0343640 A1
(43) Pub. Date: Nov. 20, 2014

(54) SYSTEMS AND METHODS FOR REDUCING BODY TEMPERATURE

(71) Applicant: Daniel Grant Ericson, Rochester, MN (US)

(72) Inventor: Daniel Grant Ericson, Rochester, MN (US)

(73) Assignee: Dynasil Biomedical Corporation, Watertown, MA (US)

(21) Appl. No.: 14/240,021

(22) PCT Filed: Aug. 23, 2012
(86) PCT No.: PCT/US12/52055

§ 371 Date: May 27, 2014

Related U.S. Application Data
(60) Provisional application No. 61/526,375, filed on Aug. 23, 2011.

(51) Int. Cl. A61F 7/10

(52) U.S. Cl. CPC A61F 7/106; A61F 2007/0057
USPC 607/105

(57) ABSTRACT

Systems and methods for lowering the body temperature of a patient or inducing hypothermia are generally described. In certain embodiments, the core body temperature of a patient can be lowered by using a heat exchanger. In certain embodiments, the heat exchanger of configured to facilitate transfer of heat from an intubation gas to a cooling fluid prior to delivering the intubation gas to the patient (e.g., to the patient's lungs).

### RELATED APPLICATIONS

This application claims priority under 35 U.S.C. §119(e) to U.S. Provisional Patent Application Ser. No. 61/526,375, filed Aug. 23, 2011, and entitled “Systems and Methods for Reducing Body Temperature,” which is incorporated herein by reference in its entirety for all purposes.

### TECHNICAL FIELD

Systems and methods for reducing the body temperature or inducing hypothermia are generally described.

### BACKGROUND

According to a 1956 study published by the National Academy of Sciences (Dripps, R. D., Ed: The Physiology of Induced Hypothermia. Washington, D.C., National Academy of Science Publication 451, 1956), the metabolism of the human body decreases by about 8% for each degree Celsius the body's temperature is lowered, and at about 28° C., the body's metabolism is about 50% of its normal level. Reducing the body's metabolism can decrease the amount of damage that metabolically active organs (e.g., the heart, the brain, etc.) sustain during ischemic and/or hypoxic events such as heart attacks and strokes. Accordingly, deliberate lowering of body temperature (i.e., inducing hypothermia) has been used in a variety of medical procedures including heart surgery, brain surgery, spinal surgery, organ transplantation procedures, and the like.

A variety of methods for lowering body temperature and inducing hypothermia are known in the art. Known methods include, for example, applying cold cloth or sponges to the body, applying ice packs to the body, submerging the body in cold fluid, and transporting a cooled gas mixture including helium to the lungs of the patient. Despite the benefits provided by the systems and methods known in the art, additional performance enhancements would be desirable.

### SUMMARY

Systems and methods for reducing body temperature, e.g. for inducing hypothermia, are described. The subject matter of the present invention involves, in some cases, interrelated products, alternative solutions to a particular problem, and/or a plurality of different uses of one or more systems and/or articles.

In one aspect, a system for lowering the core body temperature of a patient is described. In some embodiments, the system comprises a heat exchanger comprising an intubation gas inlet fluidically connected to a source of intubation gas, and an intubation gas outlet constructed and arranged to deliver intubation gas to the patient. The system further comprises, in some embodiments, an intubation tube comprising an inlet end connected to the intubation gas outlet of the heat exchanger and an outlet end constructed and arranged to eject intubation gas into the airway of the patient. In some embodiments, the heat exchanger is constructed and arranged to cool the intubation gas, and the intubation gas outlet of the heat exchanger is positioned within 5 meters of the outlet end of the intubation tube.

In certain embodiments, a system is provided. In some embodiments, the system comprises a heat exchanger comprising an intubation gas inlet fluidically connected to a source of intubation gas, and an intubation gas outlet constructed and arranged to deliver intubation gas to the patient. In certain embodiments, the system comprises an intubation tube comprising an inlet end connected to the intubation gas outlet of the heat exchanger and an outlet end constructed and arranged to eject intubation gas into the airway of the patient. In some embodiments, the heat exchanger is constructed and arranged to cool the intubation gas, the intubation gas outlet of the heat exchanger is positioned within 5 meters of the outlet end of the intubation tube, and the system is configured for use in a method of lowering the core body temperature of a patient.

In one aspect, a method of lowering the core body temperature of a patient is described. In some embodiments, the method comprises positioning a heat exchanger within 30 cm of the mouth of the patient; and transporting an intubation gas through the heat exchanger such that the intubation gas is cooled, and at least a portion of the cooled intubation gas is transported to the lungs of the patient.

The method comprises, in some embodiments, transporting an intubation gas through a first lumen of an intubation tube and into the patient's lungs; transporting a liquid through a second lumen of the intubation tube; and atomizing the liquid at or near outlet discharge end of the second lumen such that liquid droplets are injected into the airway of the patient's lungs.

In one aspect, an intubation tube is provided. In some embodiments, the intubation tube comprises a first lumen; a second lumen; and an atomizer located at or near a discharge end of the second lumen, wherein the discharge end is configured to be inserted into an airway of a patient during use.

In some embodiments, the intubation tube comprises a first lumen; a second lumen; and a sensor integrated with the intubation tube and constructed and arranged to measure at least one of a temperature and a pressure at at least one location along the length of the intubation tube, wherein the intubation tube comprises a discharge end that is configured to be inserted into an airway of a patient during use.

The intubation tube comprises, in some embodiments, a first lumen containing and transporting a gas during use; and a second lumen containing and transporting a liquid during use, wherein a discharge end of the intubation tube is configured to be inserted into an airway of a patient during use.

In some embodiments, the intubation tube comprises a first lumen constructed and arranged to transport a first fluid; a second lumen constructed and arranged to transport a second fluid; and a third lumen constructed and arranged to transport a third fluid, wherein a discharge end of the intubation tube is configured to be inserted into an airway of a patient during use.

In certain embodiments, a liquid with a boiling point of less than about 37° C. for use in a method of lowering the core body temperature of a patient is provided. In some such embodiments, the method comprises transporting the liquid with a boiling point of less than about 37° C. to the lungs of the patient.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is an exemplary schematic illustration of a system for lowering the core body temperature of patient, according to some embodiments;

FIGS. 2A-2E are, according to certain embodiments, exemplary schematic illustrations of intubation tubes used to deliver fluid to a patient; and

FIGS. 3A-3B are exemplary schematic illustrations of a heat exchanger used to cool an intubation gas, according to some embodiments.

### DETAILED DESCRIPTION

Systems and methods for lowering the core body temperature of patient are generally described. While most of the discussion below focuses on the application of inducing hypothermia, it should be understood that certain embodiments of the invention may be used/practiced for reducing the core body temperature of a hyperthermic patient (e.g. one suffering from fever or heat stroke) as well. In certain embodiments, the core body temperature of a patient can be lowered by using a heat exchanger. In certain such embodiments, the heat exchanger may be configured to transfer heat from an intubation gas to a coolant fluid prior to delivering the intubation gas to the patient (e.g., to the patient's lungs). In other embodiments, the heat exchanger may be configured to cool the intubation gas without the use of a coolant fluid (e.g., a solid-block heat exchanger, a Peltier cooler, etc.). The heat exchanger may be constructed and arranged such that it is located relatively closely to the entrance of the exit opening of the intubation tube used to deliver the intubation gas to the patient, which can ensure that the intubation gas is not excessively re-heated prior to entering the patient's body. In some embodiments, the intubation tube used to deliver cooled intubation gas to the patient can include one or more features facilitating patient cooling. For example, the intubation tube may in certain embodiments include multiple lumens. In some embodiments, one of the lumens can be used to deliver the intubation gas, while another lumen can be used to deliver a liquid such as a refrigerant. In some embodiments, a sensor can be integrated with the intubation tube, which can be used, for example, to determine a property of the fluid (e.g., temperature and/or pressure) while the fluid is in the patient's body. In certain embodiments, the intubation tube can include an atomizer that can be used to atomize a fluid (e.g., a refrigerant) exiting the intubation tube.

The injection of cooled gas into a patient's lungs to decrease body temperature is known in the art. For example, U.S. Pat. No. 6,983,749 to Kumar et al. describes a method of lowering body temperature by transporting a cooled gas mixture including helium into the lungs of the patient. In previous cooling systems, however, heat is removed from the intubation gas using a cooling device positioned at a location relatively far away from the patient's body, which leads to substantial re-heating of the cooled fluid before it reaches the lungs, thereby decreasing the efficacy of the system. In certain embodiments of the present invention, the inventive system may be configured so that a heat exchanger can be located relatively close to the patient's body and/or the exit opening of the conduit used to deliver the intubation fluid to the patient's lungs, thereby reducing the amount of heat that re-enters the intubation fluid prior to delivery to the patient's body.

In addition, it has been discovered in the context of the present invention that typical conventional intubation tubes are not ideally suited for patient cooling, and therefore, in certain embodiments of the invention, inventive intubation tubes are provided and used. Typical conventional intubation tubes generally include a first, single-lumen conduit for intubation gas delivery and a second, separate single-lumen conduit for transporting a gas to inflate a pilot balloon used to seal the lungs from the esophagus and stomach. In one aspect of the present invention, a single conduit intubation tube comprising multiple lumens is provided. In certain embodiments, at least two of the lumens are configured to deliver fluid to a patient's lungs. In certain, typical embodiments, at least one lumen of the intubation tube is not configured to deliver a fluid to the patient, but rather is configured to transport a gas to inflate a pilot balloon used to seal the lungs from the esophagus and stomach. As mentioned, in certain embodiments, the multi-lumen intubation tube may be configured to simultaneously deliver multiple fluids via separate lumens within the single conduit to the lungs of a patient. Delivering multiple fluids to the patient via separate lumens can, for example, allow one to simultaneously deliver an intubation gas and a cooling liquid without having to mix the two phases within the intubation tube. The multi-lumen intubation tube can also be configured to include a sensor(s) used to determine a property(ies) of a fluid within the intubation tube, for example during use. By incorporating the sensor into a lumen(s) of the intubation tube, as opposed to attaching the sensor to the exterior of the intubation tube, the risk of detachment from the intubation tube is reduced and more accurate property determinations may be possible.

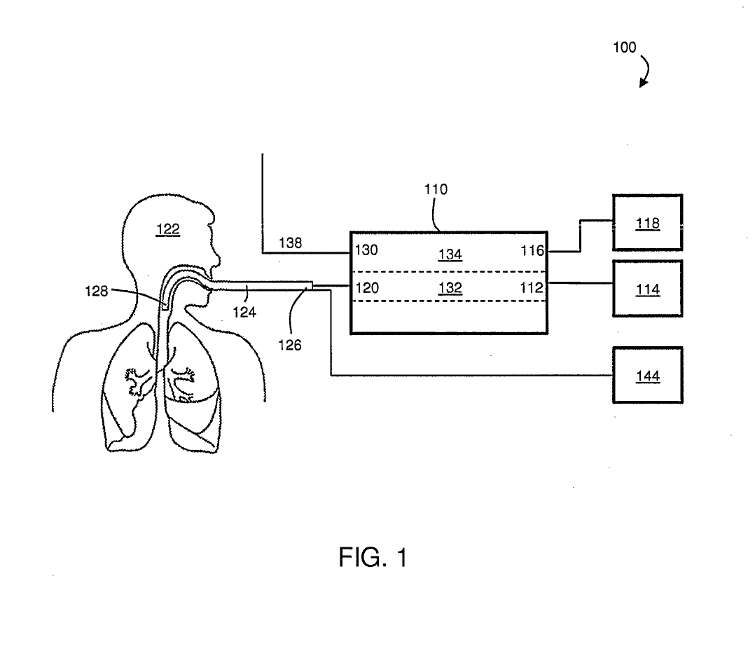

FIG. 1 is a schematic illustration of a system 100 for lowering the core body temperature of patient 122, according to some embodiments. System 100 includes heat exchanger 110 comprising an intubation gas inlet 112 fluidically connected to a source 114 of intubation gas. In addition, heat exchanger 110 includes a coolant fluid inlet 116 fluidically connected to a source 118 of coolant fluid. Heat exchanger 110 can be configured to transfer heat from the intubation gas to the coolant fluid, thereby cooling the intubation gas.

In some embodiments, the heat exchanger can be configured such that, once the intubation gas has been cooled, the intubation gas is delivered to the patient (e.g., via an intubation tube). In FIG. 1, heat exchanger 110 includes an intubation gas outlet 120 fluidically connected to inlet 126 of intubation tube 124. In some embodiments, the intubation tube comprises an endotracheal tube. In some such embodiments, the endotracheal tube can be configured to be inserted into the trachea of the patient, and the outlet end of the endotracheal tube can be configured to be positioned within the trachea of the patient during delivery of the intubation gas (and/or a liquid such as a refrigerant, as described in more detail below). For example, in FIG. 1, intubation tube 124 is configured such that outlet end 128 of intubation tube 124 ejects intubation gas into trachea and eventually the lungs of patient 122. In some such embodiments, intubation tube 124 can further comprise a balloon or other flexible material that can be inflated to seal one cavity or passageway within a patient (e.g., the lungs) from other cavities or passageways within the patient (e.g., the esophagus, the stomach, and the like). The heat exchanger may be configured such that, once the intubation gas has been cooled, the coolant fluid is transported out of the heat exchanger. In alternative embodiments, the coolant fluid may be contained under essentially non-flow conditions, for example as in a cooled fluid bath. In other embodiments, the heat absorbing media may be in solid form, such as an ice block or cooled graphite block, metal block, solid component of a Peltier cooler, etc. As an example of a flow-through heat exchanger, in FIG. 1, heat exchanger 110 includes a coolant fluid outlet 130 from which the coolant fluid used to cool the intubation gas in the heat exchanger is expelled. Optionally, after the coolant fluid is transported out of the heat exchanger, it can be transported through conduit 138, purified and/or re-cooled, and transported back to source 118 for further use in system 100. In other embodiments, the coolant fluid can be directly vented after use in heat exchanger 110.

In some embodiments, in addition to the intubation gas, intubation tube 124 can be configured to deliver a liquid. For example, in some embodiments, intubation tube 124 can be configured to transport a liquid, such as a therapeutic liquid, or, in certain embodiments a refrigerant that can be vaporized within the patient to provide an additional cooling load. In FIG. 1, liquid source 144 is fluidically connected to intubation tube 124. Liquid from liquid source 144 can be transported through intubation tube 124 and delivered to the lungs via a lumen within intubation tube 124. In some embodiments, and as described in more detail below, the lumen within intubation tube 124 that is used to deliver the liquid can be isolated from the lumen in intubation tube 124 used to deliver the intubation gas.

At least a portion of the liquid transported through the intubation tube may be atomized, in certain embodiments, prior to/upon being delivered to the patient. For example, in some embodiments, one or more atomizers positioned at or near the outlet end 128 of intubation tube 124 can be configured to atomize the liquid as it is ejected from the intubation tube. In one particular set of embodiments, the atomizers can comprise nozzles comprising 100 micrometer openings configured to produce liquid droplets (e.g., perfluorocarbon droplets) between 1 micrometer and 5 micrometers in diameter. By dispersing the liquid in small droplets prior to/upon delivering it to the patient, the speed at which the liquid is evaporated can be increased, which can lead to more rapid or effective cooling of the region of the patient to which the liquid is delivered.

While the liquid from liquid source 144 is illustrated as being delivered directly to intubation tube 124 in FIG. 1, in other embodiments, the liquid from liquid source 144 can be transported through and cooled within a heat exchanger (e.g., heat exchanger 110 and/or another heat exchanger) prior to being transported to intubation tube 124. In some such embodiments in which heat exchanger 110 is used to pre-cool the liquid from source 144, heat exchanger 110 can comprise a separate liquid inlet and a separate liquid outlet for the liquid to be delivered to the patient. In some such embodiments, the liquid can be transported through heat exchanger 110 via a separate conduit which can, for example, be surrounded by second conduit 134 of heat exchanger 110.

In some embodiments, the intubation gas outlet of the heat exchanger can be positioned relatively close to the outlet end of the intubation tube. For example, in FIG. 1, intubation gas outlet 120 of heat exchanger 110 can be positioned relatively close to outlet end 128 of intubation tube 124. Positioning the intubation gas outlet of the heat exchanger relatively close to the outlet end of the intubation tube can advantageously ensure that the intubation gas is not excessively reheated prior to being administered to the patient. In some embodiments, the intubation gas outlet of the heat exchanger is positioned within 5 meters, within 1 meter, within 50 centimeters, or within 20 centimeters of the outlet end of the intubation tube.

In certain embodiments, the heat exchanger used to cool the intubation gas can be positioned a short distance from the mouth of the patient. For example, in the set of embodiments illustrated in FIG. 1, heat exchanger 110 can be configured to be positioned a relatively short distance from the mouth of patient 122. Positioning the heat exchanger used to cool the intubation gas relatively closely to the mouth of the patient can advantageously ensure that the intubation gas is not excessively reheated prior to being delivered to the patient. In some embodiments, the heat exchanger can be positioned within 30 centimeters, within 20 centimeters, within 10 centimeters, or within 5 centimeters of the mouth of the patient.

In some embodiments, at least one of the temperature and the pressure of a fluid (e.g., the intubation gas, a refrigerant liquid, etc.) within the intubation tube can be measured, for example, prior to or as fluid is delivered to the patient. Measurement of a temperature or pressure can be achieved using, for example, one or more sensors integrated with the intubation tube, as described in more detail below. The ability to measure the temperature or pressure of a fluid being delivered to a patient can allow one to adjust upstream system parameters as necessary to provide an effective cooling load to the patient. In certain embodiments, both a temperature and pressure are able to be measured by the sensor(s).

Intubation tube 124 can include one or more inventive features. In some embodiments, the intubation tube comprises a first lumen configured for transporting the intubation gas and a second lumen (which can be fluidically isolated from the first lumen along the length of the intubation tube) configured for transporting a liquid (e.g., a refrigerant or therapeutic agent containing liquid). In certain embodiments, the intubation tube can include one or more lumens for housing sensors such as temperature sensors or pressure sensors. In some embodiments, the intubation tube can include a lumen for transporting a fluid used to inflate a pilot balloon, which can be used to seal one cavity or passageway in the patient (e.g., the lungs) from another cavity or passageway in the patient (e.g., the esophagus, the stomach, etc.).

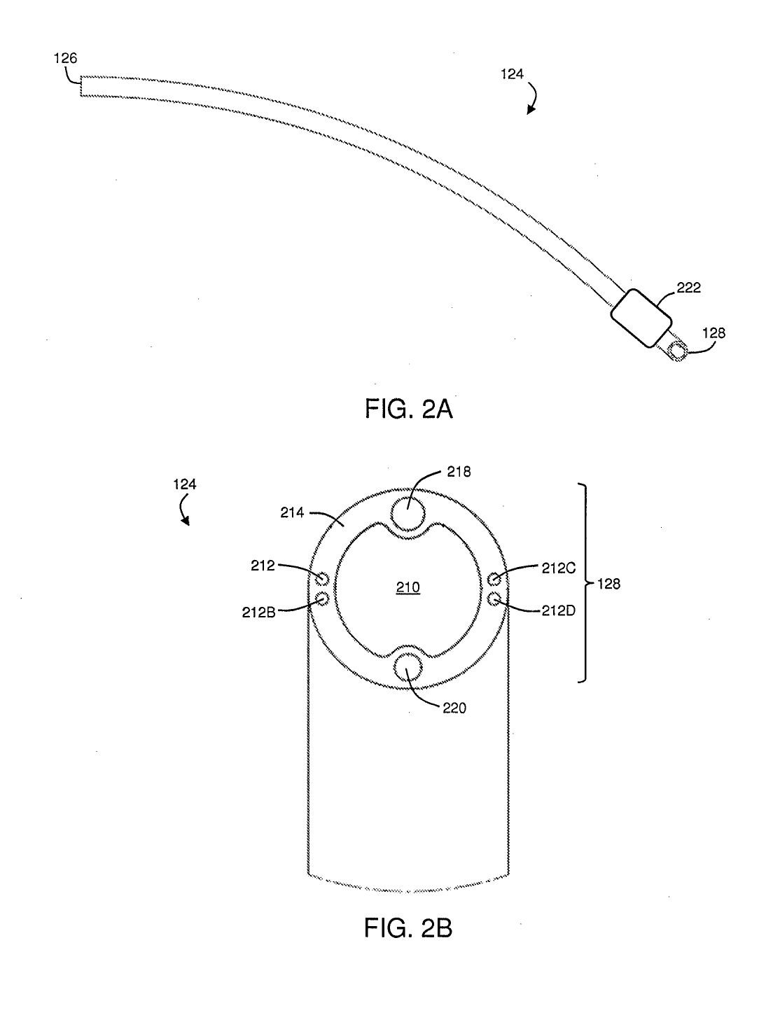

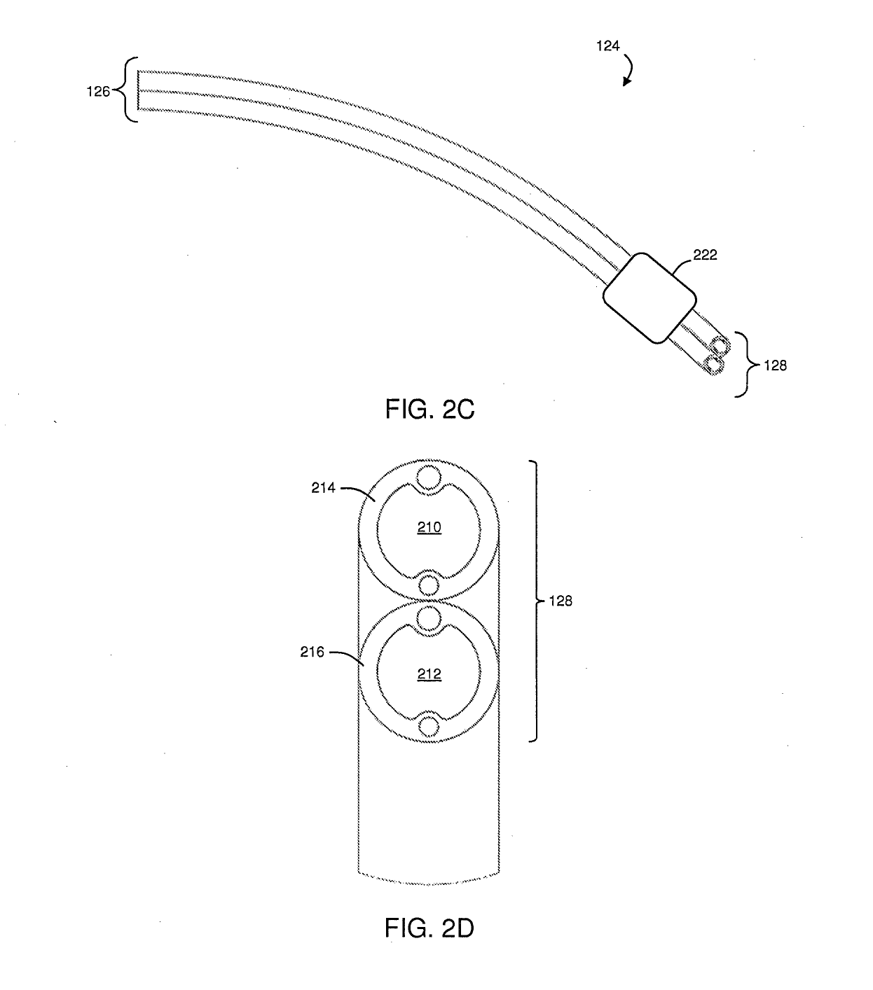

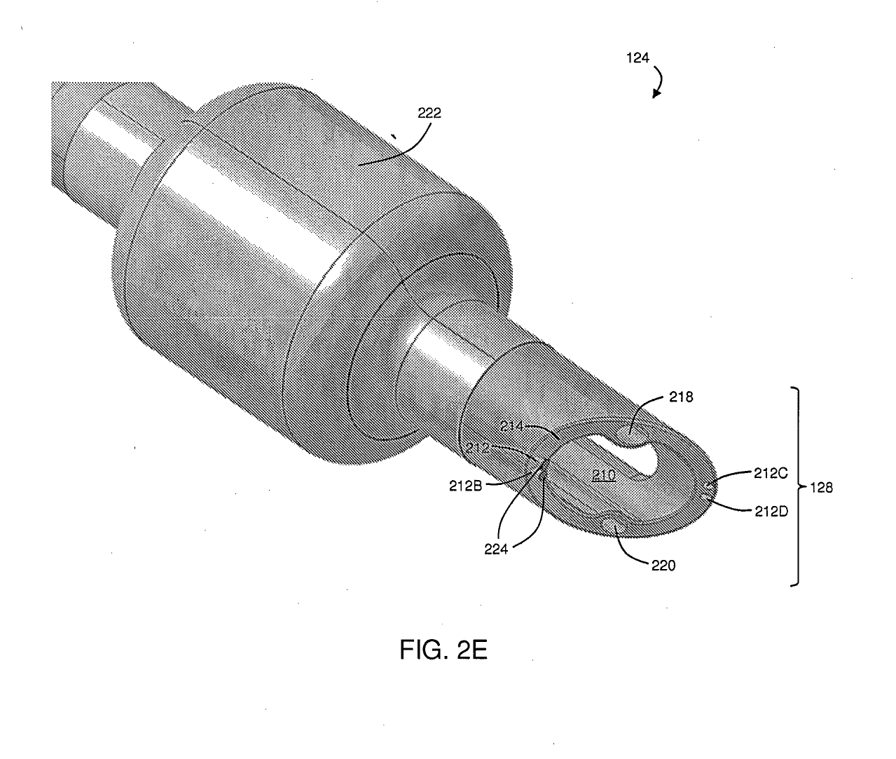

FIGS. 2A-2B are schematic illustrations of an exemplary intubation tube 124, which can be used in association with certain embodiments. FIG. 2A shows the entire length of intubation tube 124, while FIG. 2B is a close-up view of outlet end 128 of intubation tube 124. In FIGS. 2A-2B, intubation tube 124 includes first lumen 210 which can be configured to transport, for example, an intubation gas such as intubation gas from source 114 in FIG. 1. In addition, intubation tube 124 includes second lumen 212, which can be configured, for example, to transport a liquid such as a refrigerant (e.g., a liquid from source 144 in FIG. 1). Optionally, intubation tube 124 can include one or more additional lumens for transporting a liquid. For example, as illustrated in FIG. 2B, intubation tube 124 includes three additional lumens (212B, 212C, and 212D) which are configured to transport a liquid such as a refrigerant. In other embodiments, however, intubation tube 124 includes only a single lumen for transporting a liquid.

In FIGS. 2A-2B, first lumen 210 is configured as a first elongated orifice within tube body 214, and second lumen 212 is configured as a second elongated orifice within tube body 214. In other embodiments, other configurations are possible. For example, in FIGS. 2C-2D, first lumen 210 is configured as an elongated orifice within a first tube body 214, and second lumen 212 is configured as an elongated orifice within a second tube body 216 associated with the first tube body. In FIGS. 2C-2D, first tube body 214 is in contact with second tube body 216. First and second tube bodies 214 and 216 in FIGS. 2C-2D can be formed as separate tube bodies and subsequently joined, or they can be formed as a single unitary joined body.

Referring back to the set of embodiments illustrated in FIGS. 2A-2B, intubation tube 124 can optionally comprise a third lumen 218. In FIGS. 2A-2B, third lumen 218 is configured as a third elongated orifice within tube body 214. Third lumen 218 can be configured to house, for example, a sensor(s). The sensor(s) can be configured to measure at least one of a temperature and a pressure, for example, of a fluid within intubation tube 124. In some embodiments, the sensor(s) can be positioned within the intubation tube such that the sensor is within the patient during use of the intubation tube, which can allow, for example, one to measure a temperature and pressure of a fluid in the intubation tube during use.

As one example, the sensor within lumen 218 can comprise a thermocouple configured to measure a temperature of a fluid within intubation tube 124. Other examples of temperature sensors that can be positioned within third lumen 218 include, but are not limited to, resistive temperature sensors, infrared sensors, bimetallic devices, change of state sensors, and the like. Examples of pressure sensors that can be positioned within third lumen 218 include, for example, piezoresistive strain gauges, capacity pressure sensors, electromagnetic pressure sensors, piezoelectric pressure sensors, optical pressure sensors, potentiometric pressure sensors, resonant pressure sensors, thermal pressure sensors, and the like. In some embodiments, electrochemical sensors (e.g., pH sensors), fiber optic sensors, and/or glucose sensors can be positioned within a lumen of the intubation tube. While a single lumen for housing a sensor is illustrated in FIGS. 2A-2B, in other embodiments, one or more additional lumens can be incorporated into the intubation tube, which can allow for the simultaneous placement of multiple sensors (e.g., multiple temperature sensors, multiple pressure, and or a combination of one or more temperature sensors and one or more pressure sensors).

In some embodiments, intubation tube 124 comprises fourth lumen 220. Fourth lumen 220 can be configured to transport a gas for inflating a balloon or other flexible member (e.g., 222 in FIGS. 2A, 2C, and 2E) for sealing a first cavity or passageway in the patient (e.g., the lungs) from another cavity or passageway in the patient (e.g., the stomach, esophagus, etc.). For example, as illustrated in FIG. 2E, balloon 222 can be integrated with intubation tube 124 to seal the lungs of the patient from other openings within the body. Balloon 222 can be inflated by a gas or other suitable fluid that is transported through fourth lumen 220 into intubation tube 124.

An atomizer can be located near the outlet end of intubation tube 124, in some embodiments. The atomizer can be positioned relatively closely to the discharge end of a lumen (e.g., second lumen 212) used to transport a liquid (e.g., a refrigerant) through the intubation tube. For example, in some embodiments, an atomizer is positioned within 10 centimeters, within 5 centimeters, within 2 centimeters, within 1 centimeter, within 5 millimeters, within 1 millimeter, or substantially at the end of a lumen (e.g., second lumen 212 in FIGS. 2A-2E) used to transport a liquid (e.g., refrigerant) through the intubation tube. In FIG. 2E, atomizers 224 are positioned at the end of lumens 212 and 212B.

The intubation tubes described herein can be manufactured using a variety of methods. For example, in some embodiments, the intubation tube can be formed by extruding a material, such as a polymeric material, through a die to produce one or more tubes with multiple lumens. In some embodiments, multiple tubes can be attached (e.g., adhered or bonded). In some embodiments, first and second materials can be co-extruded such that the first material occupies the space defined by the material body and the second material occupies the space defined by the lumens. The second material can then be removed from the co-extruded body to form the final intubation tube structure. The intubation tubes described herein can be fabricated, in some embodiments, using hot melt tunneling, by forming a material (e.g., a melted polymer) over pre-positioned sensors or tubes, or any other methods known to those of ordinary skill in the art.

The material body of the intubation tube can be formed using a variety of materials. For example, in some embodiments, the material body of the intubation tube comprises one or more polymers (e.g., polyurethane, silicone, poly(vinyl chloride), polypropylene, polyethylene, polyesters, and/or polyamides), metals (e.g., copper, aluminum, and the like), or combinations of two or more of these materials.

Referring back to FIG. 1, heat exchanger 110 can assume a variety of configurations. In some embodiments, heat exchanger 110 comprises a first conduit 132 and a second conduit 134. In some embodiments, the first conduit 132 of heat exchanger 110 is disposed within the second conduit 134 of heat exchanger 110, for example in a shell and tube arrangement. In some embodiments, multiple conduits are disposed within second conduit 134 in a shell and tube arrangement. In some embodiments, second conduit 134 is configured such that it the longitudinal axis of second conduit 134 is substantially parallel to the longitudinal axes of the conduits contained within it (e.g., first conduit 132).

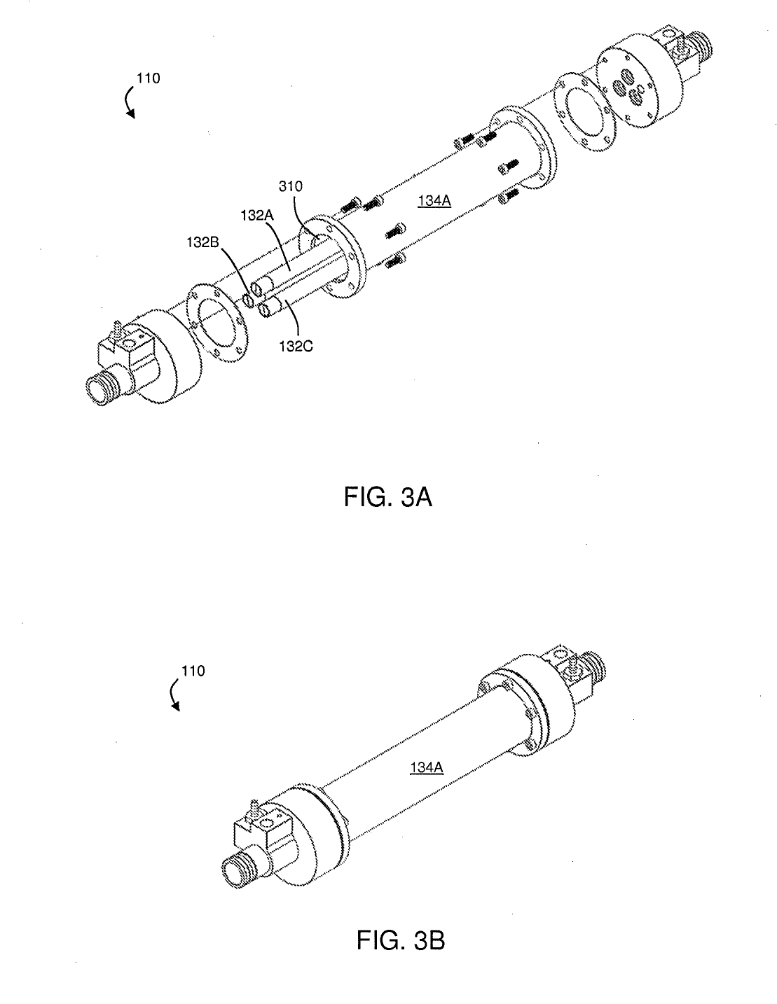

FIGS. 3A-3B are schematic illustrations of an exemplary heat exchanger 110, which can be used to transfer heat from an intubation gas (and, in some cases, a liquid such as a refrigerant) to a coolant fluid. FIG. 3A is a schematic of a disassembled heat exchanger, while FIG. 3B is a schematic illustration of the assembled heat exchanger. As illustrated in FIG. 3A, heat exchanger 110 includes a plurality of inner conduits 132A, 132B, and 132C, each of which is disposed within outer conduit 134A to form a shell and tube heat exchanger. In FIG. 3A, the longitudinal axes of each of inner conduits 132A, 132B, and 132C are substantially parallel to the longitudinal axis of outer conduit 134A. The heat exchanger can be configured, in some cases, such that intubation gas is transported through conduits 132A, 132B, and 132C while cooling fluid is transported through outer conduit 134A (e.g., via inlet 310). In other cases, the heat exchanger can be configured such that cooling fluid is transported through conduits 132A, 132B, and 132C while intubation gas is transported through outer conduit 134A. In addition, in some embodiments, the heat exchanger can be configured such that a liquid (e.g., a refrigerant) is transported through at least one of conduits 132A, 132B, 132C, and/or 134A.

The conduits of the heat exchanger can be formed from a variety of materials. In some embodiments, the inner conduits include materials with relatively high thermal conductivities to enhance the rate at which heat is transferred between the coolant fluid and the intubation gas. For example, all or part of the inner conduits can be formed of a metal or metals such as aluminum, copper, steel (e.g. stainless steel), titanium, alloys of these or other metals, and the like.

While three inner conduits are illustrated in FIG. 3A, it should be understood that, in other embodiments, more or fewer inner conduits may be present. For example, in some embodiments, the heat exchanger may comprise a single inner conduit or two inner conduits housed within a single outer conduit. In some embodiments, the heat exchanger may comprise at least 4, at least 5, at least 10, or more inner conduits housed within an outer conduit.

Fluid may be transported through heat exchanger 110 according to a variety of configurations. In some embodiments, the intubation gas in the coolant fluid can be flowed through heat exchanger 110 in a co-current flow configuration. In other embodiments, the coolant fluid and the intubation gas can be transported through the heat exchanger 110 in a counter-current configuration. In addition, one or more baffles, fins, or other fluid-directing components may be integrated into one or more conduits within heat exchanger 110 to direct the flow of fluid.

The intubation gas delivered to the patient can comprise a variety of components. In some embodiments, the intubation gas comprises air or simulated air (i.e., a mixture of oxygen and nitrogen with an oxygen/nitrogen ratio of approximately a 20:80). In some embodiments, the intubation gas can be supplemented with helium, while in other embodiments, the intubation gas does not contain supplemental helium.

The heat exchanger coolant fluid can also include a variety of components. In some embodiments, the heat exchanger coolant fluid can comprise a liquid such as, for example, polyethylene glycol, methanol, glycerol, propylene glycol, ammonia, chlorofluorocarbons, hydrochlorofluorocarbons, and hydrofluorocarbons. In other embodiments, the heat exchanger coolant fluid can comprise a gas (e.g., helium, oxygen, nitrogen, sulfur dioxide, and/or mixtures of these (e.g., air) or a liquefied gas (e.g. liquefied nitrogen).

A variety of liquids (e.g., from liquid source 144) may be transported through the intubation tube for delivery to the patient. In some embodiments, the liquid delivered to the patient comprises a refrigerant. In some embodiments, the liquid has a boiling point of less than about 37° C. For example, the liquid may comprise a perfluorocarbon. Examples of suitable perfluorocarbons with boiling points of less than about 37° C. include, but are not limited to, perfluoropropane (C₃F₈), and perfluorobutane (C₄F₁₀), perfluoropentane (C₅F₁₂). The use of refrigerants with relatively low boiling points can be advantageous, as the liquids can be delivered to the patient in the form of a liquid and evaporated within, for example, the lungs, where the gasified liquid can be subsequently exhaled. In addition, in embodiments in which the refrigerant is evaporated within the patient, the latent heat of vaporization of the refrigerant can supply an additional cooling load to the patient. However, the invention is not limited to the use of liquids with low boiling points, and, in some embodiments, liquids with boiling points of greater than about 37° C. can be transported through the intubation tube and delivered to the patient. Exemplary liquids with boiling points of greater than about 37° C. that can be transported through the intubation tube and delivered to the patient include, but are not limited to, perfluorohexane (C₆F₁₄) and the like.

While use of system 100 illustrated in FIG. 1 has been described primarily for use with a human patient, it should be understood that in other embodiments non-human patients can be used. For example, systems such as those described and outlined in FIG. 1 can be used on animals such as dogs, cats, horses, cows, pigs, and the like.

While the intubation tubes illustrated in FIG. 2A-2E have been described primarily for use in association with the systems and methods for lowering the core body temperature of a patient, as described elsewhere herein, use of the intubation tubes described herein is not so limited, and one of ordinary skill in the art would recognize that the intubation tubes described herein can be used in a variety of other systems and for a variety of other purposes, particularly where it is advantageous to deliver both a gas and a liquid to the airway of a patient and in situations where pressure or temperature monitoring of a fluid delivered to the patient is desired.

The articles, systems, and methods described herein can be used in association with a variety of procedures in which it is useful to lower the body temperature of a patient. For example, the articles, systems, and methods described herein can be used to reduce the adverse impacts of reduced oxygen availability during a variety of ischemic events including, but not limited to, cardiac arrest, stroke, traumatic brain or spinal cord injury, neurogenic fever, and neonatal encephalopathy. The articles, systems, and methods described herein can also be used to treat, for example, heat stroke.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system for lowering the core body temperature of a patient, comprising:
a heat exchanger comprising:
an intubation gas inlet fluidically connected to a source of intubation gas, and
an intubation gas outlet constructed and arranged to deliver intubation gas to the patient; and
an intubation tube comprising an inlet end connected to the intubation gas outlet of the heat exchanger and an outlet end constructed and arranged to eject intubation gas into the airway of the patient,
wherein:
the heat exchanger is constructed and arranged to cool the intubation gas, and
the intubation gas outlet of the heat exchanger is positioned within 5 meters of the outlet end of the intubation tube.

2. (canceled)

3. A system as in claim 1, wherein the heat exchanger comprises a coolant fluid inlet fluidically connected to a source of coolant fluid, and wherein the heat exchanger is constructed and arranged to transfer heat from the intubation gas to the coolant fluid.

4. A system as in claim 1, wherein the patient is a human patient.

5. A system as in claim 1, wherein the intubation gas outlet of the heat exchanger is positioned within 1 meter of the outlet end of the intubation tube.

6. A system as in claim 1, wherein the intubation gas outlet of the heat exchanger is positioned within 50 cm of the outlet end of the intubation tube.

7. A system as in claim 1, wherein the intubation gas outlet of the heat exchanger is positioned within 20 cm of the outlet end of the intubation tube.

8. A system as in claim 1, wherein the intubation gas comprises air.

9. A system as in claim 8, wherein the intubation gas comprises air supplemented with helium.

10. A system as in claim 1, wherein the intubation gas does not contain supplemental helium.

11. A system as in claim 1, wherein the intubation tube comprises an endotracheal tube constructed and arranged to be inserted into the trachea of the patient.

12. A system as in claim 1, wherein the heat exchanger comprises a first conduit and a second conduit.

13. A system as in claim 12, wherein the longitudinal axis of the first conduit is substantially parallel to the longitudinal axis of the second conduit.

14. A system as in claim 12, wherein the first conduit is disposed within the second conduit.

15. A system as in claim 1, wherein the intubation tube comprises a first lumen fluidically connected to the intubation gas outlet of the heat exchanger and a second lumen fluidically connected to a source of a liquid.

16. A system as in claim 15, wherein the liquid has a boiling point of less than about 37° C.

17. A system as in claim 16, wherein the liquid has a boiling point of less than about 37° C. comprises a perfluorocarbon.

18. A method of lowering the core body temperature of a patient, comprising:
positioning a heat exchanger within 30 cm of the mouth of the patient; and
transporting an intubation gas through the heat exchanger such that the intubation gas is cooled, and at least a portion of the cooled intubation gas is transported to the lungs of the patient.

19. A method as in claim 18, further comprising transporting a coolant fluid to the heat exchanger, wherein during the step of transporting the intubation gas through the heat exchanger, heat is transferred from the intubation gas the coolant fluid to produce the cooled intubation gas.

20. A method as in claim 18 or 19, comprising positioning the heat exchanger within 20 cm of the mouth of the patient.

21-28. (canceled)

29. An intubation tube, comprising:
a first lumen;
a second lumen; and
an atomizer located at or near a discharge end of the second lumen, wherein the discharge end is configured to be inserted into an airway of a patient during use.

30-56. (canceled)
